(19) United States
(12) Patent Application Publication
(10) Pub. No.: US 2006/0289668 A1
(43) Pub. Date: Dec. 28, 2006

(54) Scent dispersing and storage apparatus and mehtod

(76) Inventors: Joseph Szymczak, Phoenix, AZ (US); Gary Buckland, Buchanan, MI (US); John Poehner, Niles, MI (US)

Correspondence Address: WEISS & MOY PC, 4204 NORTH BROWN AVENUE, SCOTTSDALE, AZ, 85251, US

(21) Appl. No.: 11/145,361

(22) Filed: Jun. 3, 2005

(51) Int. Cl. A24F 25/00; A61L 9/04; B05B 9/08; A62C 15/00

(52) U.S. Cl. 239/36; 239/152; 239/37

(57) ABSTRACT

A scent dispersing apparatus and method includes a source of animal scent within a flexible bladder. In one embodiment, the bladder is inserted within a pouch, which pouch may be coupled to a person's leg or to an object. The source of animal scent, which may be urine, is released from the bladder, preferably in a controlled manner utilizing a valve, for purposes of leaving an animal scent along a trail or creating a scent bomb.

### FIELD OF THE INVENTION

This invention relates generally to the attraction of animals and storage of scent for hunting or other purposes and, more particularly, to an apparatus and method for the dispersion and storage of animal scents to attract animals.

### BACKGROUND OF THE INVENTION

Animal scents are frequently utilized in order to attract animals, whether for purposes of hunting or for other purposes. In the field of hunting, for example, the urine of a an animal of the type that is being hunted, for example deer urine, may be dispersed within the hunting area, in a direction leading to the hunter's “blind” or the like, to lure prey in the direction of the hunter. The idea behind such use, of course, is that animals will tend to go where they believe that other members of their species may be found.

Where the scent source is urine, it may be desired to disperse it on the ground over an extended area, so that a passing animal might sniff the scent and then follow it to the desired point. In addition to urine, a liquid food attractant may also be used. Typically, this is accomplished through the use of a “drag,” which is usually a piece of urine-saturated, absorbent material attached to the end of a string, which is dragged by a hunter on the way to his or her stand. It can be seen that the dispersal of urine through such a method is imprecise, and the drag may become tangled and may otherwise be inconvenient to use.

It may also be desired to create a scent “bomb,” i.e., a source of scent that transmits odor through the air, and that will attract animals to come in the direction of the source of the scent.

A typical source of scent for such use is animal urine packaged in a bottle. To utilize such urine, it is necessary to open the bottle and to then disperse the urine in a desired manner. However, the act of opening the bottle causes air to enter its interior, beginning a spoilage process. In addition, the use of a bottle can be messy, and some hunters prefer to use rubber gloves or the like to prevent spillage on a hunter's hands or equipment. Finally, the manipulation of a bottle, gloves and other scent-dispersing paraphernalia can be noisy, which is undesirable in a hunting setting.

A need therefore exists for a scent dispersing and storage apparatus and method that provides for improved dispersion of urine, including dispersion that is cleaner, quieter, and that limits or prevents urine spoilage by allowing for scent storage between hunts. Such an apparatus and method should permit dispersion onto the ground or in the form of a scent bomb.

The present invention satisfies these needs and provides other, related, advantages.

### SUMMARY OF THE INVENTION

In accordance with a first embodiment of the present invention, a scent dispersing apparatus is provided. The apparatus comprises, in combination: a flexible bladder adapted to hold a source of animal scent; a nozzle coupled to the bladder; wherein the animal scent passes through the exit nozzle; and an attachment member adapted to couple the flexible bladder to an object.

In accordance with another embodiment of the present invention, a scent dispersing apparatus is provided. The apparatus comprises, in combination: a flexible bladder adapted to hold a source of animal scent; a nozzle coupled to the bladder; a bladder-holding pouch; an attachment member adapted to couple the bladder-holding pouch to an object; wherein the object is one of a person's leg and a tree; and a valve regulating flow of the source of animal scent from an interior of the bladder.

In accordance with a further embodiment of the present invention, a method for dispersing an animal scent is provided. The method comprises: inserting a source of animal scent into a flexible bladder through a nozzle coupled to the bladder; attaching the flexible bladder to an object; and permitting the animal scent to exit the flexible bladder through the nozzle.

### BRIEF DESCRIPTION OF THE DRAWINGS

FIG. 1 is a front view of a scent dispersing apparatus consistent with an embodiment of the present invention.

FIG. 2 is a front, exploded view of a scent dispersing apparatus consistent with an embodiment of the present invention.

FIG. 3 is a front view illustrating a step in a process of filling a scent dispersing apparatus consistent with an embodiment of the present invention.

FIG. 4 is a perspective view illustrating a step in a process of filling a scent dispersing apparatus consistent with an embodiment of the present invention.

FIG. 5 is a perspective view illustrating a step in a process of filling a scent dispersing apparatus consistent with an embodiment of the present invention.

FIG. 6 is a perspective, exploded view illustrating a component portion of a scent dispersing apparatus consistent with an embodiment of the present invention.

FIG. 7 is a perspective, partially exploded view of the component shown in FIG. 6.

FIG. 8 is an exploded, front view illustrating components of a scent dispersing apparatus consistent with an embodiment of the present invention.

FIG. 9 is a front view of the scent dispersing apparatus of FIG. 8, in an assembled condition.

FIG. 10 is a perspective, phantom view of the scent dispersing apparatus of FIG. 9.

FIG. 11 is a side view, illustrating a use of a scent dispersing apparatus consistent with an embodiment of the present invention.

FIG. 12 is a front view, illustrating a use of a scent dispersing apparatus consistent with an embodiment of the present invention.

### DETAILED DESCRIPTION OF THE PREFERRED EMBODIMENTS

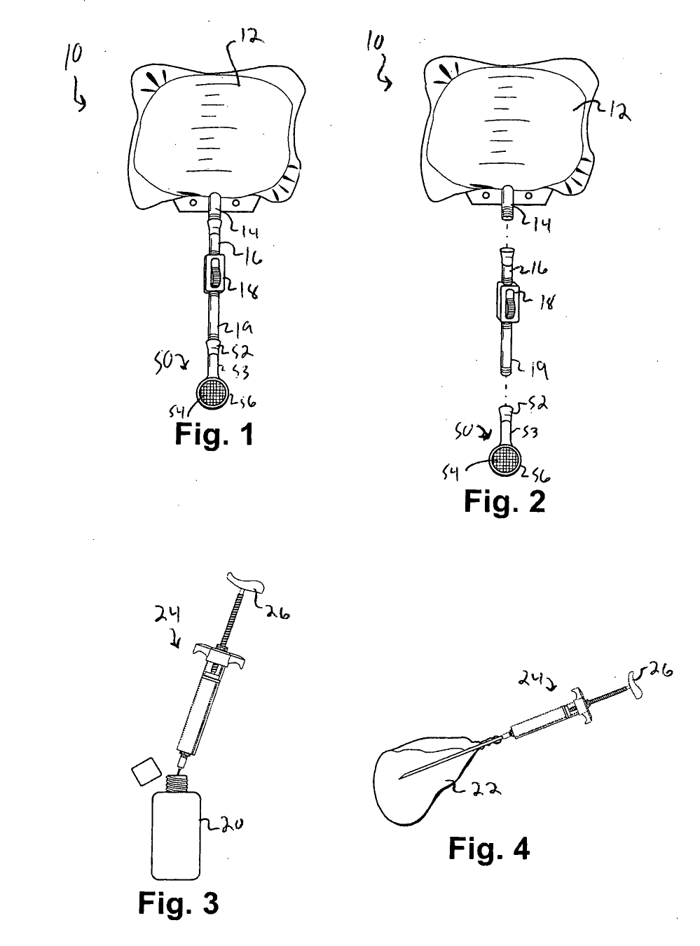

Referring first to FIGS. 1-2, an embodiment of a scent dispensing apparatus 10 is shown. The components of the dispensing apparatus 10, in this embodiment, include a flexible bladder 12, a nozzle 14, a tube 16, and a valve 18 located on the tube 16.

The bladder 12 is preferably impermeable to the passage of fluid. It is adapted to retain therein a suitable scent producing liquid. In one embodiment, the liquid is urine. Because of the impermeability of the bladder 12, the liquid may only enter or exit the bladder 12 by way of the nozzle 14. Additionally, the impermeability of the bladder 12 should substantially prevent the entry of air therein, so as to resist spoilage of the liquid.

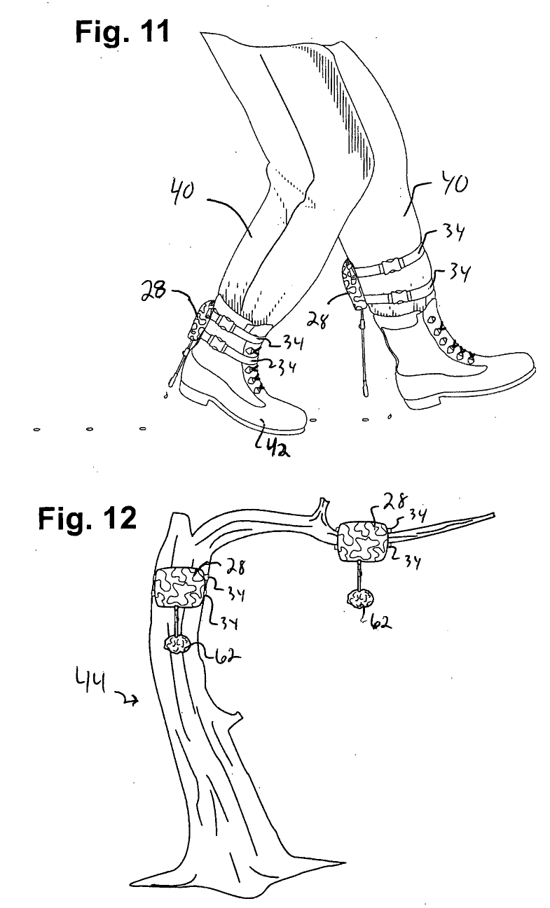

The volume of the bladder 12 may be varied as desired, depending on the use to which the apparatus 10 may be put. A volume of about four ounces may be preferred for certain hunting uses, particularly where the apparatus 10 is to be worn on a leg 40 of a user, as illustrated in FIG. 11.

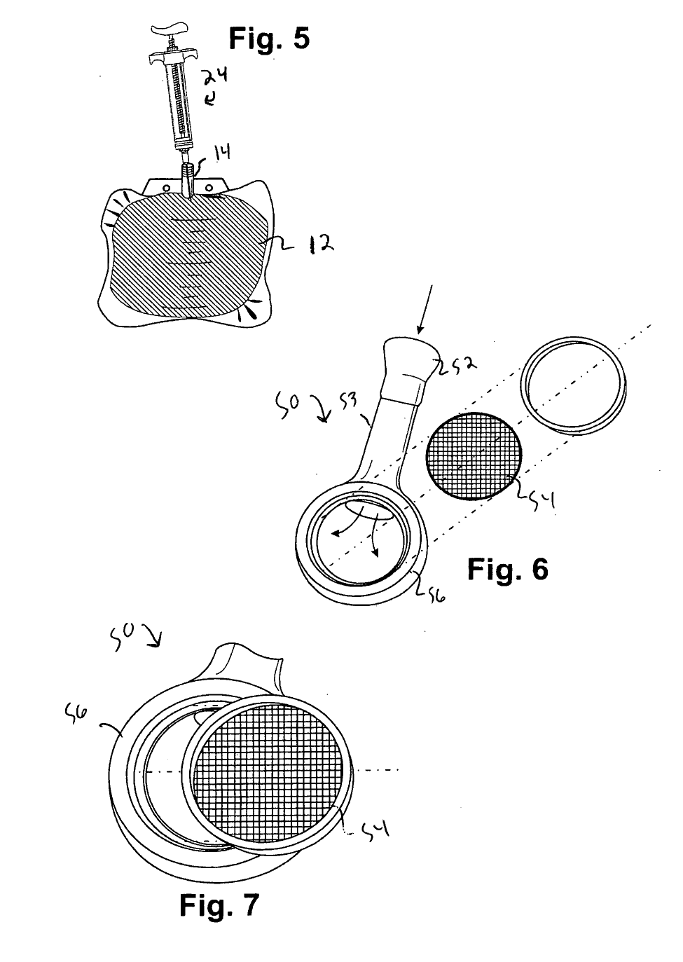

Referring now to FIGS. 3-4, the source of liquid for the bladder 12 may be, for example, a bottle 20 or an animal bladder 22. Preferably, a syringe 24 is utilized to extract the liquid from its receptacle. The syringe 24 should resist leaking, and preferably utilizes a plunger 26 that may be locked into position for ease of storage. As can be seen in FIG. 5, the syringe 24 may then be used to insert the liquid from the bottle 20, animal bladder 22 (or other container) into the bladder 12.

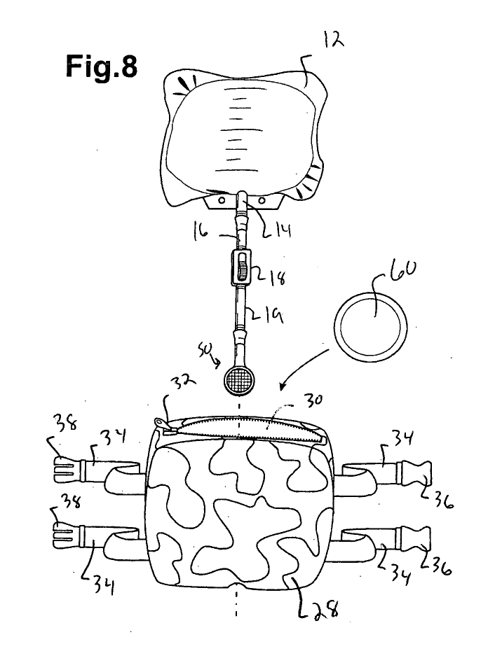

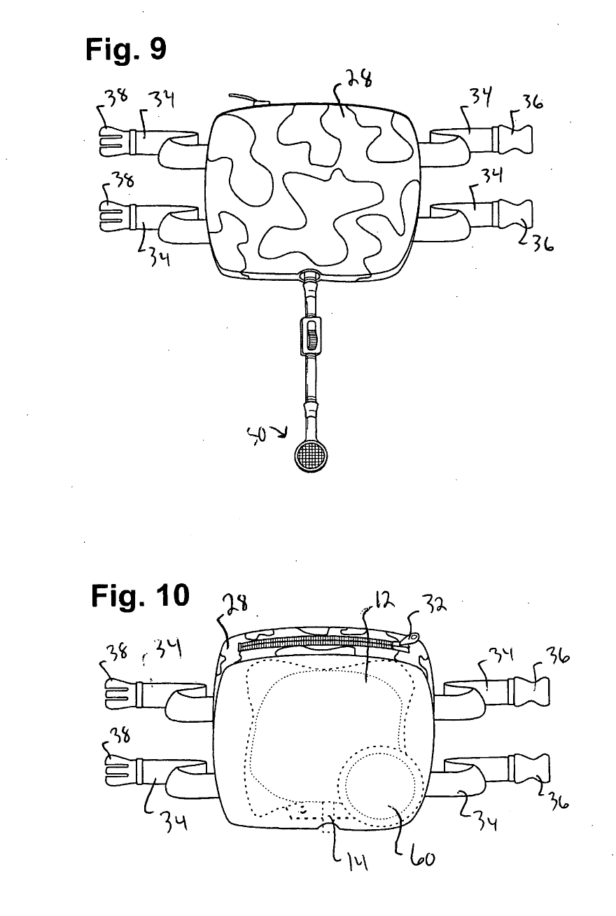

Referring now to FIGS. 8-10, in one embodiment, the bladder 12 may be inserted into a pouch 28. The pouch 28 may, as shown in FIGS. 8-9, have a camouflage type of appearance. In this embodiment, the pouch 28 has an opening 30 through an upper portion thereof, dimensioned to permit insertion of the bladder 12 therethrough, and into the interior of the pouch 28. In this embodiment, the opening 30 therethrough is closeable with zipper 32. (It should be noted that closing means, other than a zipper 32, may be utilized as desired.)

The pouch 28 may further incorporate an attachment member, to facilitate the coupling thereof to an object. In the embodiment shown in FIGS. 8-10, the attachment member comprises two sets of straps 34, having mating female buckling members 36 and male buckling members 38 thereon. It may be desired, for reasons explained in more detail below, a heating element 60, insertable into pouch 28 together with the bladder 12. The heating element 60 may be rechargeable, or may be disposable, and based on an exothermic chemical reaction.

Referring now to FIG. 11, it can be seen that the pouch 28 may be coupled to a leg 40 of a person, utilizing the straps 34 and the buckling members. In this embodiment, a first pouch 28 is coupled to a first leg 40, and a second pouch 28 is coupled to a second leg 40. (It can be seen that it may be desired to couple the pouch 28 around a boot 42, or higher along the leg 40.) Turning now to FIG. 12, one or more pouches 28 may be coupled to a tree 44—again utilizing the straps 34 and associated buckling members.

It should be noted that it may be desired to provide straps 34 of adjustable length. It may also be desired to provide other attachment means, including for example, a sleeve. The buckling members may be substituted for hook and loop fasteners or the like, though generally it will be desired to utilize means that will not unduly cause noise that might scare away an animal being sought.

Referring now to FIGS. 1-2 and 6-9, it may be desired to couple to the bladder 12—preferably though not necessarily by way of valve 18—a scent bomb element 50. The scent bomb element 50 may comprise a nipple connection 52, to facilitate coupling to the nozzle 14 extending from the bladder 12 or to a nozzle 19 extending from the valve 18. The scent bomb element 50 may further comprise an absorbent disk 54, which is preferably retained within disk frame 56. The disk 54 is preferably replaceable and biodegradable. As illustrated in FIG. 6, a tube 53 facilitates the flow of scent/liquid from the nipple connection 52 to the disk 54.

### Statement of Operation

Initially, it will be appropriate to fill the bladder 12 with a source of animal scent, which will typically, though not necessarily, be urine. The source of scent may be extracted from a bottle (FIG. 3), animal bladder (FIG. 4) or other location, and inserted into the bladder 12 (FIG. 5). A syringe 24 may be utilized for this purpose, though other means could be provided.

In one embodiment, the bladder 12 is inserted into the pouch 28 and coupled to the leg 40 of a user, as shown in FIG. 11. In this embodiment, the valve 18 may be set to permit a dripping of the scent source from the bladder 12 and out through nozzle 19. The valve 18 permits regulation of the drip rate, so that the source of scent is dispersed at a desired rate. The valve 18, it should be noted, should be corrosion resistant.

As shown in FIG. 11, it may be desired to provide two pouches 28—one coupled to a first leg 40 and a second coupled to a second leg 40. It may further be desired to provide a male animal scent in the first bladder 12 within the first pouch 28, and a female scent in the second bladder 12 within the second pouch 28. The use of male and female scents and their dispersal in a side by side fashion as illustrated in FIG. 11 can more precisely simulate a natural game trail, when a male and a female of a particular species could be expected to be traveling together.

The use of the heating element 60 acts to keep the source of scent warm. This is more natural, since urine is typically warm, and causes it to be more effectively dispersed in colder temperatures.

As shown in FIG. 12, it may be desired to use the apparatus of the present invention as a scent bomb. In this embodiment, the scent bomb element 50 is coupled to the nozzle 19. The valve 18 may be set as desired (or the scent bomb element 50 may be directly coupled to the nozzle 14), so that the source of animal scent is permitted to pass through tube 53 and into disk 54, where it is absorbed. As shown in FIG. 12, it may be desired to camouflage disk 54 with a covering 62, which covering 62 should be permeable to the passage of scent. It should be understood that the covering 62 may be a dye added to the disk 54 in order to camouflage the appearance of the disk 54.

As the source of scent is dissipated, the bladder 12 should collapse—keeping air out and the scent fresh. When a particular use is completed, it may be possible to preserve remaining liquid within the bladder 12 for future use. In addition, new scents may be stored in the bladder 12 to preserve freshness and provide for easy access of the scent. It should be further noted that while, in the preferred embodiment, a liquid is inserted into the bladder 12, substantial benefit could be derived from an alternative embodiment of the present invention in which the bladder 12 is provided pre-filled with a scent. In this way, the bladder 12 may also serve as a storage device for various liquids.

It should be noted that the use of scent to attract animals may have uses other than for hunting. Such use may be desired for purposes of observing animal behavior, counting animals, trapping animals for removal, and so forth. Any such use would be within the spirit and scope of the present invention.

I claim:

1. A scent dispersing apparatus comprising, in combination:
a flexible bladder adapted to hold a source of animal scent;
a nozzle coupled to said bladder;
wherein said animal scent passes through said exit nozzle; and
an attachment member adapted to couple said flexible bladder to an object.

2. The apparatus of claim 1 further comprising a bladder-holding pouch.

3. The apparatus of claim 1 wherein said object is a person's leg.

4. The apparatus of claim 1 wherein said object is fixed.

5. The apparatus of claim 4 wherein said object is a tree.

6. The apparatus of claim 2 further comprising a heating element capable of being inserted in said bladder-holding pouch together with said bladder.

7. The apparatus of claim 1 further comprising a valve regulating flow of said source of animal scent from an interior of said bladder.

8. The apparatus of claim 1 further comprising an absorbent material coupled to said nozzle and adapted to absorb said source of animal scent from an interior of said bladder.

9. A scent dispersing apparatus comprising, in combination:
a flexible bladder adapted to hold a source of animal scent;
a nozzle coupled to said bladder;
a bladder-holding pouch;
an attachment member adapted to couple said bladder-holding pouch to an object;
wherein said object is one of a person's leg and a tree; and
a valve regulating flow of said source of animal scent from an interior of said bladder.

10. The apparatus of claim 9 further comprising an absorbent material coupled to said nozzle and adapted to absorb said source of animal scent from an interior of said bladder.

11. A method for dispersing an animal scent comprising:
inserting a source of animal scent into a flexible bladder through a nozzle coupled to said bladder;
attaching said flexible bladder to an object; and
permitting said animal scent to exit said flexible bladder through said nozzle.

12. The method of claim 11 further comprising inserting said flexible bladder into a bladder-holding pouch.

13. The method of claim 11 wherein said object is a person's leg.

14. The method of claim 11 wherein said object is fixed.

15. The method of claim 14 wherein said object is a tree.

16. The method of claim 12 further comprising inserting a heating element into said bladder-holding pouch together with said bladder.

17. The method of claim 11 further comprising regulating a flow of said source of animal scent from an interior of said flexible bladder utilizing a valve coupled to said nozzle.

18. The method of claim 111 further comprising coupling an absorbent material to said nozzle so that it may absorb said source of animal scent from an interior of said flexible bladder.

19. The method of claim 11 further comprising:
inserting a source of animal scent into a second flexible bladder through a nozzle coupled to said second flexible bladder;
attaching said second flexible bladder to a second object;
permitting said animal scent to exit said flexible bladder through said nozzle;
wherein said object is a first leg; and
wherein said second object is a second leg.

20. The method of claim 19 wherein said flexible bladder has a male animal scent therein and wherein said second flexible bladder has a female animal scent therein.
